(19) United States
(12) Patent Application Publication
(10) Pub. No.: US 2020/0289408 A1
(43) Pub. Date: Sep. 17, 2020

(54) SOLID IMPLANT FORMULATION FOR DRUG DELIVERY

(71) Applicant: Andrew Mallon, Lincoln, RI (US)

(72) Inventor: Andrew Mallon, Lincoln, RI (US)

(21) Appl. No.: 16/818,918

(22) Filed: Mar. 13, 2020

Related U.S. Application Data
(60) Provisional application No. 62/818,559, filed on Mar. 14, 2019.

(51) Int. Cl. A61K 9/00; A61K 47/02

(52) U.S. Cl. CPC A61K 9/0024; A61K 47/02

(57) ABSTRACT

A biodegradable delivery device is disclosed that is implanted into patients to provide an extended release of one or more active pharmaceuticals for a therapeutic purpose. The delivery device comprises of: 1) an outer sheath that is composed of an inert biodegradable material that prevents the direct interactions of the core(s) with patient tissues and also facilitates the homogenous access of solvent to the surface area of the core to enable predictable core ingredient dissolution, depletion and safe and effective concentrations. 2) A Primary Core comprising one or more active ingredients plus excipients, including markers, that biodegrades and elutes all its ingredients before the sheath degrades. 3) A Secondary Core at the geometric center of the Primary core comprising a marker (including an active pharmacological or inactive ingredient that can be detected by bioanalysis, or a radio-opaque marker) that can signal when the secondary core is exposed, and excipients, which biodegrades and elutes its ingredients. 4) All elements have a marker excipient that renders the device observable remotely, including by x-ray detecting barium sulfate. The Secondary Core has a marker signal that can be distinguished from the Primary Core and Outer sheath.

### FIELD

The present technology is directed implantable drug delivery devices that provide an extended release of one or more active ingredients, and methods for its formulation and manufacture.

### BACKGROUND

Many patients have difficulties maintaining compliance to critical medications over extended periods of time, especially patients that are highly susceptible to non-compliance.

### SUMMARY

The present disclosure generally relates to a depot drug delivery device that is implanted into a patient to allow for sustained release of a drug or other pharmaceutically active material.

In one aspect, methods of manufacture of the device are described, which are unique and specialized to a particular formulation. Detailed illustration of the device are illustrated in FIG. 1.

In one embodiment, the overall implant can vary in size, according to the active ingredient. The larger the surface area of the implant, the greater the resultant drug concentration. The surface area required will be a function of the therapeutic index of the ingredient, thereby achieving efficacy without safety. In addition, the dimensions of the implant, including but not limited to the diameter, will determine how long the depot will continue to dissolute until there is nothing left.

In another aspect, described is an outer sheath or coating that is made from any biodegradable or non-biodegradeable biostable and inert material that is obvious to those skilled in the art as having suitable properties.

In a further embodiment, the outer sheath degrades more slowly than the core implant. Thereby, once the depot is depleted, the remaining sheath will also degrade. Importantly, the sheath material is chosen and delineated (thickness, concentration, density) in a manner that ensures it will outlast the depot and maintain its function until after the depot is depleted.

This attribute allows for one implanted depot to become depleted, then allow for a subsequent implant to be inserted, including at the same site.

This membrane has utility in separating the ingredients of the core from the tissues of the body into which the depot is implanted. This innovation prevents the development of localized toxicity reactions and provides additional structural integrity to the core.

The membrane has the additional utility in allowing for surface area to solvent interactions to occur homogenously across all or most of the implant. This addresses problems that may occur whereby parts of the implant surface become inaccessible to solvent due to idiosyncratic positioning against tissues. The membrane is thick enough to allow solvent to move laterally through it and solvate the entire surface area of the implant.

The depot within the sheath can be susceptible to idiosyncratic breakdown that is a result of many expected and unexpected events that can lead to crumbling, fracturing, cracking and other events that alter the intended dissolution of the depot. This event can cause rapid loss of the active ingredient, causing dangerous spikes in concentration locally and systemically, endangering patients. In addition, the depot can become exhausted prematurely due to accelerated and/or sporadic dissolution, leading to failures in therapeutic efficacy and expose the patients to dancer.

For example, these events can include: implants being brittle, and cracking or fracturing; implants being poorly compressed, causing crumbling; palpations of the implant by patients or others; injury to the implant site; idiosyncratic placement of the implant in tissues; pathological events, such as inflammation, fibrosis, and the like; and errors during implantation.

In order to render the depot opaque, we describe the inclusion of a radio-opaque substance such as Barium Sulfate that can be added to the mixture prior to formulation of the implant. This can be incorporated into the sheath and the core. As the implant dissolves, the barium also dissolves. Observation of the implant using techniques such as x-ray, can both identify that a patient has an implant and provide information as to the size that is remaining.

A particular utility of this approach is that a subsequent administration of another implant ‘dose’ can be provided with knowledge about the prior implant site and the extent to which the prior depot or depots have degraded. This avoids the danger of overdose that occurs when a new implant is provided when the prior implant is not yet depleted. This also avoids the need to undertake blood test to elucidate this.

One particular drawback of using implants is a lack of knowledge about the state of the implant. We have propounded aspects of this invention that address this already. However, one skilled in the art may seek to determine whether there is a functional implant by testing patient plasma samples for presence of the suspected drug depot.

However, this approach is unreliable because the amount of drug being released by a depot can be relatively similar from month to month. Thereby, just by measuring drug concentrations, one would not know if an implant that was intended to last 6 months, for example, was at post-implantation month 1, 2, 3, 4, 5, or 6 with any reliability.

The utility of opacity that we describe provides a reliable way to geometrically measure the mass of the implant and more accurately determine the time until depletion. Indeed, care-givers and others could repeatedly monitor size in order to track dissolution.

In certain embodiments, the active ingredient is a drug that in certain circumstances, becomes dangerous or interferes with the needs of the patients.

For example, consider an opioid use disorder patient treated with an implant containing Naltrexone, the mu-opioid antagonist, would need, at some point, to have the implant removed. This could occur due to an adverse reaction, as it could for any ingredient; but it could also occur for a pharmacodynamic reason. For example, a patient needs opioid pain relief due to a condition that is painful. Opioids would have limited effectiveness and would be dangerous to intentionally overdose to try to out-compete the extant naltrexone antagonism.

Without the opacity of this invention, there are serious problems that cannot be easily overcome.

In one example, the patient may not be conscious, fully conscious or compus mentis, and thereby not in a position to inform caregivers about the implant. Caregivers could use a method such as x-ray to easily and obviously detect the presence and position and mass of an implant. This information would then be critical in informing them as to its safe extraction. In emergency situations, this would be of particular utility.

In another example, a patient may have an implant containing a Psychiatric mediation for Schizophrenia, like Risperidone. However, if adverse reactions arise, care givers would need to be able to remove the implant also. Or, the patient may inadvertently be treated by multiple care givers and unbeknownst, the correct doses may be administered doubled due to the implant, resulting in dangerous and distressing side effects.

In another embodiment, the implant core is a bi-core design. Within the geometric center of the implant, there is a small secondary core.

This secondary core is placed at the core and is of a size that provides a particular utility. As noted, the implant depot will last a predetermined duration. Under optimum conditions and implant dissolution behavior, the core will be progressively dissolved until the secondary core is exposed and also dissolves.

The secondary core is composed of a marker. This marker is a material that provides a signal that the core is exposed.

In a particular embodiment, a secondary core includes active ingredients that are pharmacologically commensurate with the primary core.

For example, a Naltrexone primary core could have a Naloxone secondary core. For example, a Risperidone primary core could have an aripiprazole secondary core.

In a particular embodiment the core is comprised of a radio-opaque marker that is distinguishable from the primary core. Or the primary core lacks the same marker.

In a particular embodiment the core is comprised of a non-pharmacologically active marker that can be detected by bioanalysis or other methods.

The secondary core marker is a safe ingredient and may or may not have a pharmacological effect.

In a further embodiment this secondary core marker aspect of the invention provides a particular utility. As described previously, there are situations when the implant core can become prematurely exposed. In this situation, the depot can dissolute rapidly creating unintended concentrations higher than anticipated that are unsafe, and potentially harmful to the patient, leading to adverse events, including serious AEs in drugs that have a narrow therapeutic index between therapeutic efficacy and AEs. In addition, the depot may become depleted prematurely.

The exposure of the secondary core will result in the dissolution of the secondary core ingredients and their depletion.

In an embodiment where the primary and secondary core can be distinguished visually using observational methods, the observer will be able to see that, and how, the core(s) are being depleted and that the structural integrity of the implant has prematurely failed.

In another embodiment, the blood can be sampled and tested for presence of the core ingredient using bioanalysis. The core ingredient providing a detectable signal.

This secondary core provides insight into the structural integrity of the implant and will inform caregivers as to decision for implant removal or replacement.

This aspect also provides safety monitoring utility to ensure safety, including but not limited to regulatory submissions for approval and post-approval monitoring.

This implant design can be used for any pharmaceutically active material or combinations of materials within one implant.

In another aspect, a method of formulation and administration of a therapeutic dose of the composition in a scheduled manner to elicit a therapeutic effect, is described.

Also provided herein are process for the manufacturing of the implant. The processes include multiple stages including the formulation of the secondary core, the primary core, and the outer sheath; each with its own ingredients.

In another aspect, an administration kit is provided for implantation of these devices in a manner that will maintain their integrity and ensure proper functionality.

### BRIEF DESCRIPTION OF THE FIGURE

FIG. 1 illustrates a bi-core, ensheathed drug delivery implant, according to one embodiment.

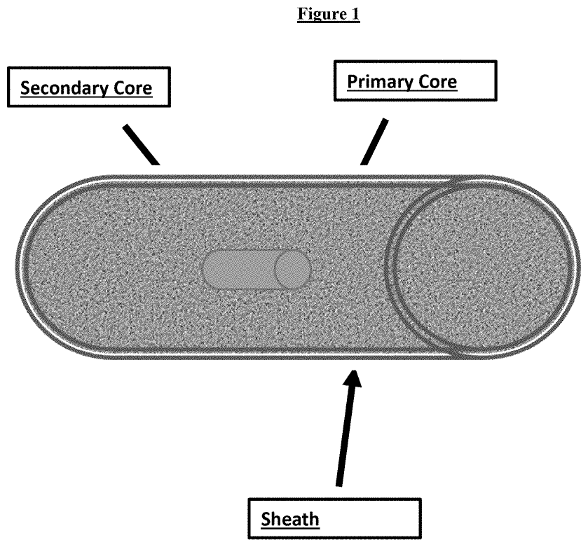

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless herein defined otherwise, all technical and scientific terms have the same meaning as is commonly understood by those of ordinary skill in the arts of this invention and as it is embodied and described. The following reference materials provide a general definition of many of the terms used in this invention: Singleton et al., Dictionary of Microbiology and Molecular Biology (2d ed. 1994); The Cambridge Dictionary of Science and Technology (Walker ed., 1988); and Hale & Marham, The Harper Collins Dictionary Of Biology (1991). While any methods, known in the art, and materials comparable or equivalent to those described herein can be used in the practice or testing of the present invention, the preferred compositions, methods and materials are described explicitly.

Compounds that may be used are those as described generally above, and are further described and drawn by the classes, subclasses, and species disclosed herein.

The dosage amount of a formulation administered to an animal or patient can be determined by physical and physiological factors such as body weight, severity of condition, the type of disease being treated, previous or concurrent therapeutic interventions, idiopathy of the patient and on the manner and/or route of administration by a practitioner skilled in the art. The skilled practitioner(s) responsible for administration will, whatever the case may be, determine the concentration of active ingredient(s) in a composition and appropriate dose(s) for the individual subject.

In certain embodiments, pharmaceutical formulations may comprise, for example, at least about 0.01% of an active compound(s). In other embodiments, the active inventive composition may comprise between about 2% to 100% of the weight of the unit, or between about 25% to about 60%, for example, and any range derivable therein.

Preferred dosage ranges include 0.001 to 20 μmol of composition per kg patient body weight. In other instances, the dosages range is from 0.005 to 5 μmol composition per kg patient body weight, optionally 0.01-5 μmol of composition per kg patient body weight. In some methods, 0.3 to 3 μmol composition per kg patient body weight are administered. In some methods, 0.1-1 μmol composition per kg patient body weight is administered, more preferably about 0.5 μmol composition per kg patient body weight. Dosage per kg body weight can be converted from rats to humans by dividing by 6.2 to compensate for different surface area to mass ratios. Dosages can be converted from units of moles to grams by multiplying by the molar weight of a composition. Suitable dosages of composition for use in humans can include 0.005 to 10 mg/kg patient body weight, or more preferably 0.02 to 5 mg/kg patient body weight or 0.1 to 1 mg/kg, or 0.2 to 0.9 mg/kg. In absolute weight for a 75 kg patient, these dosages translate to 0.075-375 mg, 0.375 to 75 mg or 7.5 mg to 75 mg or 12.5 to 67 mg. Rounded to encompass variations in e.g., patient weight, the dosage is usually within 0.5 to 500 mg, preferably 1 to 100 mg, 0.5 to 50 mg, or 1-20 mg.

In other non-limiting examples, a dose of the inventive composition and compositions may also include from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The compositions may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

Objectives, features and advantages of the embodiments shall become apparent as the description thereof proceeds when considered in connection with the accompanying illustrative drawings.

### Indications for Use

The compositions may find utility in therapeutic strategies intended to prevent, treat or cure one or more diseases, their symptoms and clinical sequelae thus arising.

In one embodiment, the subject compounds may be administered to a patient suffering from pain and/or inflammation (for example, but not limited to, arthritis, retinopathy, SLE, psoriasis, Bullous pemphigoid, shingles or a similar condition), a subject at risk of, or having undergone, microvascular insufficiency, hypoxia, sub arachnoid hemorrhage, stroke, atherosclerosis or another acute or chronic neurological ischemic events patients with mild to severe traumatic brain injury, including diffuse axonal injury, hypoxic-ischemic encephalopathy and other forms of craniocerebral trauma, patients suffering from ischemic infarction, embolism and hemorrhage, e.g., hypotensive hemorrhage, subjects with neurodegenerative diseases including Alzheimer's disease, Lewy Body dementia, Parkinson's disease, Huntington's disease, multiple sclerosis, Nieman-Pick disease, diabetic neuropathy, glaucoma, macular degeneration (wet and dry AMID), retinitis pigmentosa, motor neuron disease, muscular dystrophy, peripheral neuropathies, metabolic disorders of the nervous system including glycogen storage diseases, and other conditions where neurons are damaged or destroyed, patients with abnormal immune activation, such as autoimmune Systemic Lupus Erythematosus, rheumatoid arthritis, Bullous pemphigoid, HIV-associated disorders, Type-I diabetes, and the like; while others may include those characterized by insufficient immune function. Other diseases that may be subject to treatment with compositions of the present invention include psychiatric disorders such as attention deficit hyperactive disorder, depression (in all forms), agoraphobia, bulimia, anorexia, bipolar disorder, anxiety disorder, autism, dementia, dissociative disorder, hypochondriasis, impulse control disorder, kleptomania, mood disorder, multiple personality disorder, chronic fatigue syndrome, insomnia, narcolepsy, schizophrenia, substance abuse, post-traumatic stress disorder, obsessive-compulsive disorder, and manic depression, radiation, chemical and biological agent damage, as well as complex disorders such as Gulf War Syndrome, or such-like syndromes. Compositions of the present invention can also be used to improve outcomes regarding addiction/addiction recovery. In certain embodiments, compounds of the present invention can also be used to decrease (e.g., inhibit) cell proliferation, including but not limited to cancer. Compositions may also be used to prevent, arrest, reverse and/or treat disorders of cognition, including but not limited to autism spectrum disorders. Down's Syndrome, Angelman Syndrome, Fragile X, Alzheimer's disease, schizophrenia and ADHD.

In another embodiment, the subject compounds may be administered to a patient suffering from diseases of senescence, trauma or ischemic injuries to the brain, eye, ear, or spinal cord often produce permanent damage to neurons and cells. These injuries are serious medical problems with no effective pharmacological treatments. For example, ischemic cerebral stroke, sub-arachnoid hemorrhage or spinal cord injuries manifest themselves as acute loss of neurological capacity, encompassing small focal to global dysfunction, and sometimes leading to death. In vivo, a local decrease in CNS tissue vascular perfusion mediates neuronal death in both hypoxic and traumatic CNS injuries. Local ischemia is often caused by a disruption of the local vasculature, vessel thrombosis, vasospasm, or luminal occlusion by an, embolic mass. This ischemia is widely understood to damage susceptible neurons disrupting a variety of cellular homeostatic mechanisms and triggering apoptopic and necrotic cell death signaling events.

In certain embodiments, a method of treating or preventing a symptom associated with these diseases includes administering an effective amount of a pharmaceutical composition to a subject in need thereof, wherein the pharmaceutical composition comprises any compound of the present invention.

In some embodiments, the invention the sheath, primary and secondary core may be made of active and/or inactive pharmaceutical ingredients, and/or excipients and/or polymers.

In other embodiments, the sheath is comprised of a biodegradable polymer such as poly lactic-glycolic acid (PLGA), an alkanoid polymer, a polyhydroxyacid polymer, poly(d,l-lactic acid) polymer, or an anhydride polymer. The polymer is surface-erodible or bulk-erodible. The polymer contains a member selected from the group consisting of L-lactic acid, D-lactic acid, DL-lactic acid, malic acid, citric acid, tartaric acid, ε-caprolactone, p-dioxanone, ε-caproic acid, alkylene oxalate, cycloalkylene oxalate, alkylene succinate, β-hydroxybutyrate, substituted or unsubstituted trimethylene carbonate, 1,5-dioxepan-2-one, 1,4-dioxepan-2-one, glycolide, glycolic acid, L-lactide, D-lactide, DL-lactide, meso-lactide, and any optically active isomers, racemates or copolymers thereof.

Illustrative pharmaceutically acceptable excipients, carriers, or fillers that may be used in accordance with the various embodiments include, but are not limited to, magnesium stearate, stearic acid, starch, and cellulose.

In another aspect, provided are subcutaneously implantable implants comprising any of a number of pharmaceutically and/or biologically active substances and drugs, including, but not limited to, protein drugs such as insulin, drugs used to treat rheumatoid arthritis and other forms of arthritis, antibiotics, and antiviral substances including anti HIV vaccines, deworming and distemper drugs such as those administered to pets and/or livestock, and drugs used for treating urinary tract infections, cancer-control/antineoplastic agents, vitamins and nutraceuticals, and any other pharmaceutical/biologically active substances. In similar manner as their opiate antagonist-comprising counterparts, these inventive implants are effective to maintain the desired drug/substance levels for an extended period of time, up to and exceeding ninety days. These drug comprising implants are prepared from compressed admixtures comprising one or more drugs, an anti-inflammatory agent and a pharmaceutically acceptable carrier in the form of a subcutaneously implantable pellet.

Illustrative active ingredients includes, but is not limited to, conventional, experimental, novel, or as yet unknown pharmaceuticals, drugs, or biologically active substances. Illustrative active ingredients include, protein drugs such as insulin; desensitizing agents such as ragweed pollen antigens, hay fever pollen antigens, dust antigen and milk antigen; vaccines such as smallpox, yellow fever, distemper, hog cholera, fowl pox, antivenom, scarlet fever, diphtheria toxoid, tetanus toxoid, pigeon pox, whooping cough, influenza, rabies, mumps, measles, poliomyelitis, Newcastle disease, etc.; antiinfectives, such as antibiotics, including penicillin, tetracycline, chlortetracycline bacitracin, nystatin, streptomycin, neomycin, polymyxin, gramicidin, oxytetracycline, chloramphenicol, and erythromycin; sulfonamides, including sulfacetamide, sulfamethizole, sulfamethazine, sulfadiazine, sulfamerazine, and sulfisoxazole; anti-virals including idoxuridine; and other antiinfectives including nitrofurazone and sodium propionate; antiallergenics such as antizoline, methapyrilene, chlorophenaramine, pyrilamine and prophenpyridamine; antiallergenics such as hydrocortisone, cortisone, hydrocortisone acetate, dexamethasone, dexamethasone 21-phosphate, fluocinolone, triamcinolone, medrisone, prednisolone, prednisolone 21-phosphate, and prednisolone acetate; decongestants such as phenylephrine, naphthazoline, and tetrahydrazoline; miotics and anticholinesterases such as pilocarpine, eserine salicylate, carbachol, diisopropyl fluorophosphate, phospholine iodide, and demecarium bromide; such as atropine sulfate, cyclopentolate, homatropine, scopolamine, tropicamide, eucatropine, and hydroxyamphetamine; sympathomimetics such as epinephrine; sedatives and, hypnotics such as pentobarbital sodium, phenobarbital, secobarbital sodium, codeine, (a-bromoisovaleryl)urea, carbromal; psychic energizers such as 3-(2 aminopropyl)indole acetate and 3-(2 amino butyl) indole acetate; Tranquilizers such as reserpine, chloropromaline, and thiopropazate; androgenic steroids such as methyltestosterone and fluorymesterone; estrogens such as estrone, 17 B-estradiol, ethenyl estradiol, and diethyl stilbesterol; progestational agents such as progesterone, megestrol, melengestrol, chlormadinone, ethisterone, norethynodrel, 19-nor-progesterone, norethindrone, medroxyprogesterone and 17 B-hydroxy-progesterone; Humoral agents such as the prostaglandins, for example PGE1, PGE2, and PGF2; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine; antihistamines such as diphenhydramine, dimenhydrinate, tripelennamine, perphenazine, and chlorophenazine; cardioactive agents such as dibenzohydroflumethiazide, flumethiazide, chlorothiazide, and aminotrate; nutritional agents such as vitamins, essential amino acids and essential fats, and veterinary pharmaceuticals.

### EXAMPLES

### Example 1

A patient is admitted to the emergency room following an automobile accident. The patient has suffered traumatic injuries and is distressed and in considerable pain. Physicians administer an opioid analgesic intravenously to ease the pain but it has no effect. The physician suspects that the patient may be under the influence of a drug such as naloxone or naltrexone that is blocking the effects of the opioid. The physician knows where implanted drugs are usually placed and x-rays are performed that identify a mass in the abdomen consistent with an active implant releasing Naltrexone. The physician removes the implant. Shortly thereafter, the patient responds to opioid pain medication.

### Example 2

A patient suffering from Schizophrenia is admitted to the hospital suffering from lethargy and tachycardia. The patient informs physicians that he has an implant that contains Risperidone with an inner core of aripiprazole that was implanted 1 month ago and should last another 2 months. Suspecting that there is an overdose of Risperidone, the physicians request blood plasma samples and order xrays of the implant site to create a 3D observation. The x-rays show that the implant has cracked along the center and crumbled. Lame amounts of Risperidone have already eluted from the implant and only 20% of the implant mass remains. The secondary core, which is brighter due to a higher concentration of contrast agent shows sign of depletion. Blood tests show that as well as high levels of Risperidone, there is also aripiprazole present in plasma. The implant still has structural integrity due to the sheath and is removed under local anesthesia. The patient is transferred to Risperidone tablets at the correct dose level and the side effects quickly dissipate. Physicians discover that the patient experienced a minor trauma at the implant site 2 weeks ago that may have caused the implant to crack.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. An implanted biodegradable drug delivery device for the controlled release of one or more active pharmaceuticals over an extended period of time to produce local or systemic effects, the device comprising an inert, longer-lasting biodegradable outer sheath that separates a primary core and a secondary core from a patient's tissues, and which does not substantially biodegrade or lose its functionality or structural integrity until after the primary or secondary core has been depleted.

2. The device of claim 1, wherein the sheath is opaque to observations using technologies such as x-ray by way of excipients such as barium sulfate addended to the ingredients.

3. The device of claim 1, wherein the primary core comprises one or more pharmaceutically active ingredients, and one or more pharmaceutically acceptable excipients, including an excipient that provides opacity, such as Barium Sulfate.

4. The device of claim 1, wherein the secondary core is disposed at the geometric center of the primary core, the secondary core containing a marker that alerts an observer that the core is exposed, where the marker can include a pharmacodynamically equivalent active ingredient, or non-active or active ingredient that can be detected, including by plasma bioanalysis; or a radio-opaque marker that can be distinguished from the primary core and sheath marker, including by altering the relative concentration of the markers.

5. A manufacturing method comprising of steps required to create a bi-core pellet comprising the Primary and Secondary Core and the ensheathing process; and the preparation of the ingredients and their sterilization.

6. An administration method and device for the safe, effective and proper administration and implantation of the device.
